(19) United States
(12) Patent Application Publication
(10) Pub. No.: US 2025/0281391 A1
(43) Pub. Date: Sep. 11, 2025

(54) EFFERVESCENT COMPOSITION AND METHOD FOR DETERMINING BITTERNESS PERCEPTION

(71) Applicant: BAYER HEALTHCARE LLC, Indianola, PA (US)

(72) Inventors: Tejas KEKATPURE, Edison, NJ (US); Devon SEWELL, Dover, NJ (US); Debanjan DAS, Morristown, NJ (US)

(21) Appl. No.: 19/071,165

(22) Filed: Mar. 5, 2025

Related U.S. Application Data
(60) Provisional application No. 63/562,021, filed on Mar. 6, 2024.

(51) Int. Cl. A61K 9/46; A61K 9/20; A61K 31/192; A61K 31/485; A61K 33/10; A61K 33/14; A61K 33/30

(52) U.S. Cl. CPC A61K 9/0007; A61K 9/2009; A61K 9/2013; A61K 31/192; A61K 31/485; A61K 33/10; A61K 33/14; A61K 33/30

(57) ABSTRACT

An effervescent composition is provided herein that includes an effervescent combination including sodium bicarbonate and citric acid to taste mask the bitter taste perception of various molecules or compositions, such as an active pharmaceutical ingredient or a composition including an active pharmaceutical ingredient. Also provided is a method of reducing bitterness perception of a molecule or composition by adding the effervescent combination; and a method of determining bitterness perception of a molecule or a composition by measuring electric potential and optionally adjusting the bitterness perception of the molecule or composition by adding the effervescent combination.

### FIELD

This disclosure generally relates to an effervescent combination including sodium bicarbonate and citric acid, compositions including the effervescent combination, and methods for reducing the bitter taste perception of bitter tasting molecules and compositions. Also disclosed are in vitro methods for determining bitterness perception of bitter tasting molecules and compositions using electric potential measurements.

### BACKGROUND

This section provides background information related to the present disclosure which is not necessarily prior art.

When formulations enter the mouth or nose, molecules interact with saliva and bind to taste receptors in the oral cavity (i.e., on the tongue, soft palate, upper esophagus, check, and epiglottis). Taste bud receptors recognize the molecules and send perceptions of taste to the brain. Many molecules/compositions are known to elicit an unpleasant and/or bitter taste perception in humans. It happens that many of these molecules/compositions are drugs or active pharmaceutical ingredients (APIs) that must be in oral dosage forms, such as tablets, liquid solutions, or nasal sprays to treat or prevent different ailments. Because some of these molecules/compositions have a bitter taste, it is desirable to reduce the perception of their unpleasant and/or bitter tastes.

Current methods to mask unpleasant taste perception, or to make molecules and/or compositions more palatable or taste-neutral, include partially removing the molecules/compositions eliciting an unpleasant taste perception, coating or microencapsulating them, generating effervescence reactions, and/or adding taste masking substances. Coating or microencapsulating methods are not suitable for some molecules/compositions, such as inorganic salts, as they may need to dissociate in solutions for case of formulation adsorption when taken orally. There are many taste masking substances available, but achieving an acceptable level of taste masking usually requires the use of considerable amounts of the taste masking substance. This requires additional costs, and often, the addition of the taste masking substances can leave additional unpleasant taste perception(s).

Additionally, detection and quantification of bitterness perception for oral formulations is an ongoing challenge in product development. Often, taste testing becomes a qualitative exercise using human subjects (or human tongues) which are extremely variable in response. In addition, some people are sensitive or allergic to product tasting, thus human subjects may not be suitable for testing. Most importantly, quantitative evaluation is not possible in human taste tests, and the bitterness perception is scaled arbitrarily in relative subjective terms such as “more bitter” and “less bitter” when comparing two compositions. Other methods include using electronic tongues to measure and compare tastes. Such instruments use “taste sensors” to receive information from chemicals on the tongue and use pattern recognition to detect tastes that compose the human palate, such as sourness, saltiness, bitterness, sweetness, and umami (savoriness).

Despite the commercial availability of methods for masking taste, there is a need in the art for new and improved compositions and methods for masking and detecting bitter taste.

### SUMMARY

This section provides a general summary of the disclosure and is not a comprehensive disclosure of its full scope or all of its features.

In certain aspects, the present disclosure provides an effervescent composition including an effervescent combination, a molecule or composition imparting a bitter taste, and one or more pharmaceutically acceptable excipients. The effervescent combination may include a ratio of about two to about three moles sodium bicarbonate to about one mole citric acid, and the molecule or composition imparting a bitter taste may be an active pharmaceutically ingredient (API) or a composition comprising an API. In further aspects, the API may be an inorganic salt, such as calcium carbonate, zinc sulphate, magnesium chloride, or a combination thereof. In other aspects, the API may be an organic salt, such as dextromethorphan hydrobromide or naproxen sodium. In some aspects, the pharmaceutically acceptable excipient may be a sweetener, a flavoring agent, or a lubricant.

In some aspects, the effervescent composition is in an oral dosage form, such as a solid oral dosage form comprising a tablet, powder, granule, or an extruded form, such as a rod. In other aspects, the effervescent composition is a liquid dosage form, such as a solution.

In further aspects, the effervescent composition can have a positive electric potential of at least about 5 mV when dissolved in 100 mL water and measured at about 25° C. In further aspects, the effervescent combination can be present in the effervescent composition in an amount to increase the electric potential of the composition by at least about 15 mV when the composition is dissolved in 100 mL water and measured at 25° C. compared to the composition dissolved in 100 ml of water at 25° C. without the effervescent combination. Additionally or alternatively, the effervescent combination can be present in the composition in an amount from about 20% to about 40% by weight of the composition.

The present disclosure also provides a method of reducing bitterness perception of a molecule or composition that includes adding an effervescent combination to the molecule or composition. In a particular embodiment, the effervescent combination added may comprise a ratio of about three moles sodium bicarbonate to about one mole citric acid. The method may further include adding the effervescent combination to the molecule or composition in an amount to increase the electric potential of the molecule or composition by at least about 15 mV when dissolved in 100 mL water and measured at 25° C. compared to the molecule or composition when dissolved in 100 mL water and measured at 25° C. without the effervescent combination.

In yet other aspects, the present disclosure provides a method of determining bitterness perception of a molecule or composition, such as an API or a composition including an API. In certain aspects, the method includes measuring electric potential of the molecule or composition, wherein the molecule or composition is dissolved in water, and wherein as electric potential increases bitterness perception of the molecule or composition decreases. Additionally, in some further aspects, the method may further comprise adjusting the bitterness perception of the molecule or composition by adding an effervescent combination comprising citric acid and sodium bicarbonate to the molecule or composition.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a table showing electric potential (mV) and pH readings for various solutions. All solutions contain 100 mL de-ionized water. Additionally, some solutions contain various weights of the effervescent couple (sodium bicarbonate and citric acid). Additionally or alternatively, some solutions contain different combinations and weights of molecules or compositions that impart a bitter taste (e.g., APIs or compositions comprising APIs), such as calcium carbonate, zinc sulfate, magnesium chloride, dextromethorphan hydrobromide (sometimes abbreviated Dex. HBr), naproxen sodium, acetaminophen, guaifenesin, and caffeine.

FIG. 2 is a bar chart showing the electric potential (mV) for solutions containing calcium carbonate, zinc sulfate, and magnesium chloride with and without the effervescent combination.

FIG. 3 is a bar chart showing the electric potential (mV) for solutions containing zinc sulfate with and without the effervescent combination.

FIG. 4 is a bar chart showing the electric potential (mV) for solutions containing magnesium chloride with and without the effervescent combination.

FIG. 5 is a bar chart showing the electric potential (mV) for solutions containing Dex. HBr with and without the effervescent combination.

FIG. 6 is a bar chart showing the electric potential (mV) for solutions containing naproxen sodium with and without the effervescent combination.

### DETAILED DESCRIPTION

### A. Introduction

An effervescent combination including sodium bicarbonate and citric acid to reduce the bitterness perception of bitter tasting molecules and compositions, such as active pharmaceutical ingredients (APIs) and compositions comprising APIs, is disclosed herein.

Although citric acid is known to suppress bitter tastes, it has not been combined with sodium bicarbonate for taste masking, especially not at a molar ratio of about 2:1 to about 3:1 of sodium bicarbonate to citric acid. Some current methods of reducing the perception of bitterness involve using insoluble salts or forming insoluble complexes of the ions. Using the effervescent combination described herein allows salts to be soluble instead of making them insoluble to mask their taste. This allows for taste masking without changing the natural state of bitter molecules/compositions.

Without being bound by theory, the inventors have discovered that increasing the electric potential of a bitter tasting molecule/composition with the effervescent combination results in a decrease in bitter taste perception of inorganic and organic molecules.

Additionally described herein are methods of determining bitterness perception of a molecule or composition, and optionally adjusting the bitterness perception by addition of the effervescent combination. The novel methods solve the problem of subjective human taste tasting by accurately predicting the bitterness perception of a molecule or composition using in vitro electrochemical tests. Such methods include measuring the electric potential/degree of carbonation of the molecule or composition when dissolved in water. Electric potential measurements can be used as a surrogate to predict bitterness perception of the molecule or composition, such as an API or a composition comprising an API. In particular, once the electric potential of the molecule or composition is measured, the effervescent combination described herein can be added to reduce the electric potential, and thus reduce the perception of bitterness of the molecule or composition. Correlations were achieved with solutions prepared with incremental concentrations of different APIs and electric potential properties. Such methods can also help make formulations without conducting tests on human subjects, such as sensory analysis panels.

Example embodiments are provided so that this disclosure will be thorough and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

### B. Definitions

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this disclosure, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art. Particularly in reference to a given quantity, number or percentage, “about” is meant to encompass deviations of plus or minus ten percent (+10). For example, about 5% encompasses any value between 4.5% to 5.5%, such as 4.5, 4.6, 4.7, 4.8, 4.9, 5, 4.1, 5.2, 5.3, 5.4, or 5.5. Accordingly, unless otherwise indicated, the numerical parameters set forth in this specification and attached claims are approximations that can vary depending upon the desired properties sought to be obtained by the presently disclosed subject matter.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As will be understood by one skilled in the art, for any and all purpose, all ranges disclosed herein also encompass any and all possible subranges and combinations of subranges thereof. Furthermore, as will be understood by one skilled in the art, a range includes each individual member.

Unless defined otherwise, technical and scientific terms used herein have the same meaning as commonly understood by a person of ordinary skill in the art. In particular, this disclosure utilizes routine techniques in the field of taste masking and taste perception.

The term “active pharmaceutical ingredient” or “API” as used herein refers to any molecule, composition, formulation, compound, ingredient, or substance that is an active component in a pharmaceutical drug that prevents, diagnosis, treats, or cures disease in a mammal. APIs may be “over-the-counter” and not require a drug prescription, while some may require a drug prescription from a medically-authorized person. Some APIs described herein may impart an unpleasant and/or bitter taste perception.

### C. Effervescent Composition

In one embodiment, an effervescent composition including an effervescent combination of sodium bicarbonate and citric acid is provided herein. The effervescent combination can be used to reduce the bitterness perception of a molecule or composition by raising the electric potential when both the effervescent combination and molecule or composition imparting a perceived bitter taste are dissolved together in water.

An effervescent combination refers to any combination of ingredients/components that produce carbon dioxide in a liquid and may mask a perceived unpleasant taste of a molecule or composition. Non-limiting examples of perceived unpleasant tastes include bitter, sour, pungent, astringent, cardboardy, dusty, dry, floury, rancid, and metallic tastes and/or aftertastes. In some embodiments, the effervescent combination comprises sodium bicarbonate and citric acid. The effervescent combination may further comprise an additional component besides sodium bicarbonate and citric acid to help taste mask the molecule or composition. Additional taste masking components include sweetening agents, flavoring agents, or flavor-blocking agents, such as bitter flavor blockers. Bitter flavor blockers may include adenosine monophosphate, lipoproteins, or phospholipids, and may compete with the bitter molecules/compositions to bind to the G-protein coupled receptors on the tongue (receptor sites that detect bitterness), thus suppressing the bitter taste.

Thus, the effervescent combination may be referred to as a combination (including two, three, four, five, etc. components). When the effervescent combination only includes two components, it may be referred to as an effervescent couple.

### Molar Ratio of Sodium Bicarbonate to Citric Acid

Sodium bicarbonate, CAS Number 144-55-8, is a chemical compound having the IUPAC name sodium hydrogen carbonate and chemical formula NaHCO₃. It is a salt composed of a sodium cation (Na⁺) and a bicarbonate anion (HCO₃⁻). It is commonly known as baking soda. Sodium bicarbonate or calcinated sodium bicarbonate (heated sodium bicarbonate to remove moisture making it more anhydrous) may be used herein.

Citric acid, CAS Number 77-92-9, is an organic compound having the IUPAC name 2-hydroxypropane-1,2,3-tricarboxylic acid and chemical formula HOC(CO₂H)(CH₂CO₂H)₂. It is a colorless weak organic acid and has two forms. Citric acid can be obtained as an anhydrous (water-free) form or as a monohydrate. In one embodiment, citric acid as used herein is citric acid, anhydrous (containing less than 1% water).

In one embodiment, the effervescent combination may be present at a particular molar ratio of sodium bicarbonate to citric acid. The molar ratio of sodium bicarbonate to citric acid may allow the effervescent combination to reduce bitterness perception via increased electric potential/degree of carbonation.

A molar ratio of about 2 to about 3 moles of sodium bicarbonate to about 1 mole of citric acid was found to mask the bitter taste perception of inorganic and organic molecules and compositions, such as APIs and compositions comprising APIs. Thus, in a particular embodiment, the effervescent combination may be present in a molar ratio of about 2:1 to about 3:1 moles of sodium bicarbonate to citric acid.

As used herein about 2 moles of sodium bicarbonate may refer to a range of about 1.5 to 2.5 moles, 1.6 to 2.5 moles, 1.7 to 2.5 moles, 1.8 to 2.5 moles, 1.9 to 2.5 moles, 2.0 to 2.5 moles, 2.1 to 2.5 moles, 2.2 to 2.5 moles, 2.3 to 2.5 moles, 2.4 to 2.5 moles, 1.5 to 2.4 moles, 1.5 to 2.3 moles, 1.5 to 2.2 moles, 1.5 to 2.1 moles, 1.5 to 2.0 moles, 1.5 to 1.9 moles, 1.5 to 1.8 moles, 1.5 to 1.7 moles, 1.5 to 1.6 moles, 1.6 to 2.4 moles, 1.7 to 2.3 moles, 1.8 to 2.2 moles, 1.9 to 2.1 moles, 1.8 to 2.0 moles, or 1.95 to 2.05 moles. For example about 2 moles of sodium bicarbonate may be about 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, or 2.5 moles of sodium bicarbonate.

As used herein about 3 moles of sodium bicarbonate may refer to a range of about 2.5 to 3.5 moles, 2.6 to 3.5 moles, 2.7 to 3.5 moles, 2.8 to 3.5 moles, 2.9 to 3.5 moles, 3.0 to 3.5 moles, 3.1 to 3.5 moles, 3.2 to 3.5 moles, 3.3 to 3.5 moles, 3.4 to 3.5 moles, 2.5 to 3.4 moles, 2.5 to 3.3 moles, 2.5 to 3.2 moles, 2.5 to 3.1 moles, 2.5 to 3.0 moles, 2.5 to 2.9 moles, 2.5 to 2.8 moles, 2.5 to 2.7 moles, 2.5 to 2.6 moles, 2.6 to 3.4 moles, 2.7 to 3.3 moles, 2.8 to 3.2 moles, 2.9 to 3.1 moles, 2.8 to 3.0 moles, or 2.95 to 3.05 moles. For example about 3 moles of sodium bicarbonate may be about 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, or 3.5 moles of sodium bicarbonate.

As used herein, about 1 mole of citric acid may refer to a range of 0.5 to 1.5 moles, 0.6 to 1.5 moles, 0.7 to 1.5 moles, 0.8 to 1.5 moles, 0.9 to 1.5 moles, 1.0 to 1.5 moles, 1.1 to 1.5 moles, 1.2 to 1.5 moles, 1.3 to 1.5 moles, 1.4 to 1.5 moles, 0.5 to 1.4 moles, 0.5 to 1.3 moles, 0.5 to 1.2 moles, 0.5 to 1.1 moles, 0.5 to 1.0 moles, 0.5 to 0.9 moles, 0.5 to 0.8 moles, 0.5 to 0.7 moles, 0.5 to 0.6 moles, 0.6 to 1.4 moles, 0.7 to 1.3 moles, 0.8 to 1.2 moles, 0.9 to 1.1 moles, 0.8 to 1.0 moles, or 0.95 to 1.05 moles of citric acid. For example, about 1 mole of citric acid may be 0.5, 0.55, 0.6, 0.65, 0.7, 0.75, 0.8, 0.85, 0.9, 0.95, 0.96, 0.97, 0.98, 0.99, 1.0, 1.1, 1.2, 1.3, 1.4, or 1.5 moles of citric acid.

Each range disclosed herein includes end values. Additionally, each value disclosed herein can be part of a range with another disclosed value. Ultimately, one skilled in the art is aware that minor variations in the ratio of 3:1 can be used and still provide taste masking benefits.

For purposes of this disclosure, the molecular weight used for citric acid is 192.12 g/mol and the molecular weight for sodium bicarbonate is 84.01 g/mol. Additionally, for purposes of this disclosure, the amount of moles of a compound is calculated by dividing the grams of a compound by the molecular weight of that compound.

For example, the following calculation is used to determine the moles of citric acid (CA) in 0.25 grams of CA:

| 0.25 g CA | 1 mole CA |
|---|---|
| 1 | 192.12 g CA |

=0.25 g/192.12 g = 0.001301 mol CA

Additionally, for example, the following calculation is used to determine the moles of sodium bicarbonate (SB) in 0.25 grams of SB:

| 0.25 g SB | 1 mole SB |
|---|---|
| 1 | 84.01 g SB |

=0.25 g/84.01 g = 0.002976 mol SB

In some embodiments, the number of moles can be brought out to 4 significant figures.

Thus, the molar ratio of SB:CA in this example is 0.002976:0.001301. Molar ratio may be simplified to the “n:1” form. For example, the following calculation may be used to determine the molar ratio in “n:1” form:

$$\frac{0.002976}{0.001301} = \frac{x}{1} ; x = 2.2873 ; thus the SB : CA molar ratio is 2.2873 : 1$$

### Amount of Effervescent Combination in Composition

Further, the effervescent combination may be present in the effervescent composition in any amount sufficient to reduce the perception and/or impression of bitterness of one or more bitter tasting molecule or composition, such as an API or a composition comprising an API.

For example, in one embodiment, the effervescent combination may be present in the effervescent composition in an amount of about 10% to about 20%, about 20% to about 30%, about 30% to about 40%, about 40% to about 50%, about 50% to about 60%, about 1% to about 50%, about 5% to about 45%, about 10% to about 40%, and about 20% to about 40% weight by weight (% w/w) of the effervescent composition. The effervescent combination may be present in the effervescent composition in an amount of about 15% to about 45% w/w, about 16% to about 44% w/w, about 17% to about 43% w/w, about 18% to about 42% w/w, about 19% to about 41% w/w, about 20% to about 40% w/w, about 21% to about 39% w/w, about 22% to about 38% w/w, about 23% to about 37% w/w, about 24% to about 36% w/w, about 25% to about 35% w/w, about 26% to about 34% w/w, or about 27% to about 33% w/w of the effervescent composition. In a particular embodiment, the effervescent combination may be present in the effervescent composition in an amount of about 27.5% w/w of the effervescent composition.

In a particular embodiment, sodium bicarbonate and citric acid may each be present in the effervescent composition in an amount from about 125 mg to about 300 mg, particularly about 150 mg to about 250 mg, such as about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg. In a further particular embodiment, the sodium bicarbonate and citric acid are present in substantially the same weight amount in the effervescent composition. For example, about 150 mg: 150 mg sodium bicarbonate to citric acid or about 200 mg: 200 mg sodium bicarbonate to citric acid or about 250 mg: 250 mg sodium bicarbonate to citric acid.

Similarly, each ingredient/component in the effervescent combination (sodium bicarbonate, citric acid, etc.) may be present in the effervescent composition in an amount of about 10% to about 20%, about 20% to about 30%, about 30% to about 40%, about 40% to about 50%, about 50% to about 60%, about 1% to about 50%, about 5% to about 45%, about 10% to about 40%, and about 20% to about 40% weight by weight (% w/w) of the effervescent composition.

In a further embodiment, the effervescent composition can have a positive electric potential. For example, the electric potential may be at least about 5 mV, about 10 mV, about 15 mV, about 20 mV, about 25 mV, about 30 mV, about 35 mV, about 40 mV, about 45 mV, about 50 mV, about 60 mV, about 70 mV, about 80 mV, about 90 mV, about 100 mV, about 150 mV, about 200 mV, or about 300 mV when dissolved in water. In a particular embodiment, the electric potential may be about 5 mV when dissolved in water. The water the effervescent combination is dissolved in may be at a temperature of about 5° C., about 10° C., about 15° C., about 20° C., about 25° C., about 30° C., about 35° C., about 40° C., about 45° C., or about 50° C. In a particular embodiment, the electric potential may be measured at a water temperature of about 25° C. The effervescent composition may be dissolved in an amount of water of about 10 mL, about 20 mL, about 30 mL, about 40 mL, about 50 mL, about 100 mL, about 250 mL, about 500 mL, and about 1000 mL to measure electric potential. In a particular embodiment, the effervescent composition can be dissolved in about 100 ml of water to measure electric potential. In some embodiments, about 0.1, about 0.15, about 0.2, about 0.25, about 0.3, about 0.35, about 0.4, about 0.45, about 0.5, about 0.55, about 0.6, about 0.65, about 0.7, about 0.85, about 0.9, about 0.95, about 1.0 grams, about 1.25 grams, about 1.5 grams, about 1.75 grams, about 2.0 grams, about 2.25 grams, about 2.5 grams, about 2.75 grams, about 3 grams, about 3.25 grams, about 3.5 grams, about 3.75 grams, about 4 grams, about 4.25 grams, about 4.5 grams, about 4.75 grams, or about 5 grams of the effervescent composition can be dissolved in about 100 ml of water. In a particular embodiment, about 0.3 grams of the effervescent composition can be dissolved in about 100 mL of water. In another particular embodiment, about 0.4 grams of the effervescent composition can be dissolved in about 100 mL of water. In another particular embodiment, about 0.5 grams of the effervescent composition can be dissolved in about 100 mL of water. In another particular embodiment, about 1.5 grams of the effervescent composition can be dissolved in about 100 mL of water.

Thus, in another embodiment, the effervescent combination may be present in the effervescent composition in an amount to increase the electric potential of the effervescent composition. For example, the effervescent combination may be present in the effervescent composition in an amount to increase the electric potential of the effervescent composition by at least about 5 mV, about 10 mV, about 15 mV, about 20 mV, about 25 mV, about 30 mV, about 35 mV, about 40 mV, about 45 mV, about 50 mV, about 60 mV, about 70 mV, about 80 mV, about 90 mV, about 100 mV, about 150 mV, about 200 mV, or about 300 mV compared to a composition without the effervescent combination. In a particular embodiment, the effervescent combination is present in the effervescent composition in an amount to increase the electric potential of the effervescent composition by at least about 15 mV compared to a composition without the effervescent combination.

Additionally, in another embodiment, the effervescent combination may be present in the effervescent composition to allow the effervescent composition to have an average disintegration time of about 1.0 minute to about 2.5 minutes, particularly about 2.0 minutes using the standard United States Pharmacopcia (USP) 701 disintegration test (basket rack assembly, semi-automatic test apparatus.

Molecule or Composition with an Unpleasant Taste Perception

Additionally, the effervescent composition can contain one or more molecule or composition with an unpleasant taste perception, such as a perceived bitter taste, bland taste, earthy taste, or other undesirable taste. Such molecule or composition may be or contain any molecules/compositions known to impart an unpleasant/bitter perception of taste. For example, the molecule or composition may be an active pharmaceutical agent (API) or a composition comprising an API that imparts an unpleasant/bitter taste perception. The effervescent combination described herein may be used to mask the taste of any molecule or composition by raising the electric potential of the molecule or composition when dissolved in water.

In one embodiment, the unpleasant or bitter tasting molecule or composition, which may also be an API or a composition comprising an API, may be an inorganic molecule or a composition comprising an inorganic molecule. Such inorganic molecules may be acids, oxo acids, hydroxides, metal oxides, non-metal oxides, binary salts and those containing polyatomic ions, and peroxides. Non-limiting examples of inorganic minerals include aragonite, potassium, phosphorous, calcium, magnesium, and sulfur. Non-limiting examples of inorganic salts include sodium chloride, calcium chloride, potassium chloride, sodium sulfate, calcium phosphate, calcium carbonate, zinc sulfate, and magnesium chloride.

Additionally or alternatively, the unpleasant or bitter tasting molecule or composition, which may also be an API or a composition comprising an API, may be an organic molecule or a composition comprising an organic molecule, such as an organic salt. Organic molecules may be nucleic acids, lipids, sugars, proteins, enzymes, hydrocarbon fuels, and carbohydrates. Other examples include solvents, drugs, vitamins, dyes, artificial flavors, toxins, and molecules used as precursors to biochemical compounds. Non-limiting examples of organic molecules include azelastine (commonly referred to as ASTELIN®, ASTEPRO®, OPTIVAR®, etc.), dextromethorphan hydrobromide (commonly referred to as Dex. HBr, BENYLIN®, DELSYM®, etc.), naproxen sodium (commonly referred to as ALEVER, ANAPROX®, NAPROSYN®, etc.), diphenhydramine hydrochloride (commonly referred to as BENADRYL®, BANOPHEN™, etc.), acetaminophen (commonly referred to as TYLENOL®, etc.), guaifenesin (commonly referred to as GUIATUSS®, ROBITUSSIN®, etc.), caffeine, acetaldehyde, acetone, biotin, heptane, vanillin, methanol, bromophenol blue, carbon tetrachloride, fullerene, and benzaldehyde. The organic molecule may be natural or synthetic.

Each unpleasant or bitter tasting molecule or composition, which may be an API or a composition comprising an API, may be present in the effervescent composition in an amount of about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 1% to about 50%, about 50% to about 100%, about 5% to about 45%, about 10% to about 40%, about 20% to about 40%, about 55% to about 95%, about 60% to about 90%, or about 70% to about 80% weight by weight (% w/w) of the effervescent composition. The bitter tasting molecule or composition may be present in an amount of 5%, 10%, 15%, 20%, 25%, 30%, 35%, 40%, 45%, 50%, 55%, 60%, 65%, 70%, 75%, 80%, 85%, 90%, or 95% w/w. In a particular embodiment, the unpleasant or bitter tasting molecule or composition may be present in an amount of about 70.5% w/w.

### Method of Making Effervescent Composition

The effervescent composition described herein can be prepared by known methods. For example, the effervescent combination and/or bitter tasting molecule or composition (e.g., an API or a composition comprising an API) may be mixed with one or more pharmaceutically acceptable excipient/additive. Suitable additives are all customary formulation auxiliaries. Non-limiting examples of pharmaceutically acceptable excipients include sweeteners, flavoring agents, lubricants, extenders, solvents or diluents, solid carriers and fillers, surfactants, dispersants or binders or fixatives, preservatives, dyes and pigments, defoamers, inorganic and organic thickeners, water repellants, and other further processing auxiliaries. Additionally, in an effervescent solution, water may also be present, for example an effervescent solution in an enclosed container, such as a bottle.

A non-limiting list of flavoring agents includes natural flavoring agents (such as those derived from natural sources like plants, fruits, and vegetables), artificial flavoring agents (such as vanillin synthetically produced for a vanilla flavor), processed flavoring agents (such as smoked flavors, roasted flavors, and fermented flavors), masking agents (such as salt) and sweeteners. Examples of natural flavoring agents are vanilla extract, cinnamon, ginger, and citrus oils.

A non-limiting list of sweeteners includes sugar (such as sucrose, fructose, and glucose), sugar alcohol (such as erythritol and maltitol), sugar substitute (such as aspartame, sucralose, and stevia), syrups, honey, molasses, glycyrrhizin, unrefined sweetener (such as coconut sugar), and allulose).

A non-limiting list of lubricants includes solid lubricants (such as calcium stearate, magnesium stearate, talc, etc.) and liquid lubricants (such as liquid paraffin). Additional examples of lubricants include stearic acid, sodium stearyl fumarate, polyethylene glycols, and silicon dioxide.

It is possible to use colorants such as inorganic pigments. Examples of inorganic pigments include iron oxide, titanium oxide, Prussian blue, and organic dyes, such as alizarin dyes, azo dyes, and metal phthalocyanine dyes, and trace nutrients, such as salts of iron, manganese, boron, copper, cobalt, molybdenum, and zinc.

Other additives may also be present. For example, such additional components may include protective colloids, binders, adhesives, thickeners, thixotropic substances, penetrants, stabilizers, sequestering agents, and complex formers. In general, the active compounds can be combined with any solid or liquid additive customarily used for formulation purposes.

Each of the other additives may be present in the effervescent composition in an amount of about 10% to about 20%, about 20% to about 30%, about 30% to about 40%, about 40% to about 50%, about 50% to about 60%, about 1% to about 50%, about 5% to about 45%, about 10% to about 40%, and about 20% to about 40% weight by weight (% w/w) of the effervescent composition.

In a particular embodiment, the effervescent composition may comprise one or more API, two or more alkalinizing agent (effervescent combination), one or more sweetener, one or more flavoring agent, and one or more lubricant. In a particular embodiment, the effervescent composition comprises an API, an effervescent combination described herein, a sweetener, a flavoring agent and a lubricant. In a particular embodiment, the API is calcium carbonate, the alkalinizing agents are calcinated sodium bicarbonate and citric acid, the sweetener is stevia, the flavoring agent is a mixed berry flavor, such as EVOSPRAY® Natural Mixed Berry Flavor WONF, and the lubricant is magnesium stearate. Additionally, or alternatively, the flavoring agent may be an orange flavor, such as EVOSPRAY® Natural Orange Flavor WONF.

In a particular embodiment, the effervescent composition comprises about 60% to 80% w/w of an API and about 20% to about 30% of the effervescent combination. The effervescent composition may comprise about 10%, 20%, 30%, 40%, 50%, 60%, 70%, 80%, or 90% of an API, and respectively, about 90%, 80%, 70%, 60%, 50%, 40%, 30%, 20%, or 10% of the effervescent combination,

Depending on the formulation type, processing steps may be performed such as, for example, wet grinding, dry grinding, or granulation.

The composition described herein may be an oral dosage form or a nasal dosage form. The oral dosage form may be a solid oral dosage form such as a tablet, powder, pill, granule, chewing gum, candy, and an extruded form, such as a rod. In a particular embodiment, the solid oral dosage form is a tablet, powder or pill. In a particular embodiment, the solid oral dosage form is a tablet, for example a pressed tablet. In another embodiment, the solid oral dosage form, such as a pressed tablet, is capable of or configured to dissolve in the mouth (in situ) rather than having to be added to water, such as a powder formulation containing sodium bicarbonate and citric acid, which is added to water and then consumed by a subject. In some embodiments, the solid oral dosage form can dissolve in the mouth without the addition of water. In other words, in some embodiments the solid oral dosage form can dissolve in the mouth using/with only saliva.

In another particular embodiment, the solid oral dosage form is not a chewing gum nor a microparticle formulation. Indeed it was surprising that one does not need to rely on the combination of microparticles and effervescent couple comprising sodium bicarbonate and citric acid to taste mask bitter compounds. The inventors have discovered that an effervescent couple comprising sodium bicarbonate and citric acid alone in the amount described herein can suitably mask the taste of unpleasant/bitter tasting compounds without the need for microparticles.

Alternatively, the oral dosage form may be a liquid dosage form, such as a solution. The effervescent solution may be enclosed in a container, such as a bottle with a removeable cap/lid or other suitable container to not allow the effervescence to escape until use. Such solutions may be oral care compositions, such as mouthwashes, toothpastes, or tooth gels, for example; or the solutions may be liquid over-the-counter or prescription medications. The nasal dosage form may be a nasal spray, ointment, drop, or gel. The dosage form may also be a pharmaceutical product or a food product.

It is understood that the above examples are non-limiting, and for any ingestible product containing a molecule or composition causing unpleasant taste perception, it may be appropriate to add therein the effervescent combination or composition according to the present disclosure.

The effervescent composition containing sodium bicarbonate and citric acid described herein can be used in a method of reducing bitterness perception described below.

### D. Method of Reducing Bitterness Perception Using Effervescent Combination

In an additional embodiment, a method for reducing the bitterness perception of one or more molecule or composition, such as an API or a composition comprising an API, is provided. In general, the bitterness perception of a molecule or compound may be reduced by adding an effervescent combination of sodium bicarbonate and citric acid as described herein.

As used herein, the terms “molecule” and “composition” may refer to any molecule or composition that may impart an unpleasant or bitter taste perception; or any molecule or composition in which a person wants to determine if the molecule or composition has an unpleasant or bitter taste perception. Molecules or compositions, as used herein, may be or contain APIs. Additionally, the molecules or compositions described herein may be or may contain any inorganic or organic molecule. Compositions described herein may contain one or more than one distinct molecule that each impart an unpleasant or bitter taste perception. Molecules and compositions described herein may also be referred to as ingredients, compounds, substances, components, or formulations.

Without being bound by theory, the effervescent combination described herein containing sodium bicarbonate and citric acid may mask the unpleasant taste perception of bitter molecules or compositions by releasing carbon dioxide gas when in contact with water/saliva, which leads to chemical irritation of taste receptors in the mouth. Thus, the more carbonation/fizz generated from the release of carbon dioxide gas, the less bitter a molecule or composition will be perceived as. Without being bound by theory, the bitter taste stimuli will be masked by chemical irritation of the taste buds.

The molecule or composition to which the effervescent combination may be added in order to reduce perceived bitterness of the molecule or composition, may include one or more API. As discussed above, an API may be an inorganic molecule such as calcium carbonate, zinc sulfate, or magnesium chloride. Alternatively, the API may be an organic molecule such as dextromethorphan hydrobromide, naproxen sodium, acetaminophen, guaifenesin, or caffeine. The choice of bitter tasting molecule or composition, such as an API or composition comprising an API, is not particularly important as the effervescent combination can effectively taste mask a molecule or composition by raising the electric potential of the molecule or composition.

For exerting the taste masking effect, the effervescent combination can be mixed with the unpleasant tasting molecule or composition (such as an API or a composition comprising an API). Generally, both the effervescent combination and unpleasant tasting molecule/composition may be contained in an ingestible or edible product. Alternatively, the effervescent combination and unpleasant tasting molecule/composition may be combined in situ, typically followed by thorough mixing.

In one embodiment, the method of masking or reducing the unpleasant or bitter taste perception of a molecule or composition causing the unpleasant or bitter taste perception may include the step of adding the effervescent combination as described herein, to the molecule or composition causing the unpleasant/bitter taste perception. Typically, the effervescent combination is added to the molecule or composition causing the unpleasant taste perception in a taste masking effective amount.

As used herein, a taste masking effective amount is the amount that is sufficient to mask, reduce, modify, or eliminate the unpleasant taste of the unpleasantly tasting molecule or composition as compared to the molecule or composition without the effervescent combination. The taste masking effective amount may vary based on the particular unpleasant tasting molecule or composition, on its relative amount in the composition, and/or on the particular orally/nasally administered product. Those skilled in the art will have no difficulty determining the appropriate amount of the effervescent combination in each particular case. For example, the appropriate amount of the effervescent combination may be when the effervescent combination is present in a composition in an amount of about 20% to about 40% w/w.

In addition to the effervescent combination being added to the molecule or composition causing the unpleasant taste perception in a taste masking effective amount, the effervescent combination may be added in a molar ratio of sodium bicarbonate to citric acid of about 2:1 to about 3:1, as discussed above, to achieve a taste masking effect.

Additionally or alternatively, the method may include adding the effervescent combination to a molecule or composition in an amount to change (e.g., increase or decrease) the electric potential of the molecule or composition.

The electric potential of the molecule or composition may be increased by at least about 5 mV, about 10 mV, about 15 mV, about 20 mV, about 25 mV, about 30 mV, about 35 mV, about 40 mV, about 45 mV, about 50 mV, about 60 mV, about 70 mV, about 80 mV, about 90 mV, or about 100 mV when dissolved in water compared to a molecule or composition when dissolved in water without the effervescent combination. In a particular embodiment, the effervescent combination may be added to the molecule or composition in an amount to increase the electric potential by at least about 15 mV compared to a molecule or composition without the effervescent combination.

The water in which the effervescent combination and molecule or composition are dissolved in may be at a temperature of about 5° C., about 10° C., about 15° C., about 20° C., about 25° C., about 30° C., about 35° C., about 40° C., about 45° C., or about 50° C. In a particular embodiment, the electric potential may be measured at a temperature of about 25° C. The effervescent composition may be dissolved an amount of water selected from about 10 mL, about 20 mL, about 30 mL, about 40 mL, about 50 mL, about 100 mL, about 250 mL, about 500 mL, and about 1000 mL to measure electric potential. In a particular embodiment, the effervescent composition can be dissolved in about 100 mL of water to measure electric potential.

As shown in the Examples, it has been found that the effervescent combination including sodium bicarbonate and citric acid shows strong taste masking activity. Indeed, the effervescent combination can surprisingly reduce and/or mask the unpleasant/bitter taste impression of a variety of different molecules and/or compositions, such as APIs or compositions containing APIs. Additionally, the inventors discovered that one can determine the perceived bitterness of a molecule or composition by measuring the electric potential of the molecule or composition when dissolved in water, and optionally adjust the electric potential, thus adjusting the perceived bitterness of the molecule or composition (i.e., taste mask the perceived bitterness), by adding the effervescent combination to the molecule or composition as described in further detail below.

### E. Method of Determining Perceived Bitterness by Measuring Electric Potential

In a further embodiment, a method for determining perceived bitterness is provided herein. The inventors have discovered that the electric potential of a molecule or composition can be used as a proxy to determine bitterness perception of a molecule or composition.

Thus, the method may include measuring electric potential of a molecule or composition, for example a molecule with a bitter taste perception such as an API, by dissolving the molecule or composition in water. As the electric potential increases the bitterness perception of the molecule or composition may decrease.

In one embodiment, a method of determining bitterness perception of a molecule or composition is provided herein comprising measuring the electric potential of the molecule or composition, wherein the molecule or composition is dissolved in water, and wherein as electric potential increases bitterness perception of the molecule or composition decreases.

In another embodiment, the method may further comprise adjusting the bitterness perception of the molecule or composition (or the electric potential) by adding an effervescent combination comprising citric acid and sodium bicarbonate to the molecule or composition. The effervescent combination may comprise a molar ratio of about 2:1 to about 3:1 sodium bicarbonate to citric acid as described herein. The molecule or composition may comprise an API.

The change in bitterness perception may be reliant on the concentration of one or more molecule or composition imparting a bitter taste, such as an API or composition comprising an API. For example, a lower concentration of a molecule or composition imparting a bitter taste may not change the bitterness perception, while a higher concentration of a molecule or composition imparting a bitter taste may. For example, an increase in concentration of a molecule or composition imparting a bitter taste of at least 1%, 2%, 3%, 4%, 5%, 6%, 7%, 8%, 9%, 10%, 20%, 30%, 40%, 50%, 100%, 200%, etc. may result in an increase in bitterness perception.

Additionally, the perceived bitterness of a molecule or composition (or the electric potential) may be adjusted by addition of the effervescent combination (comprising citric acid and sodium bicarbonate). The perceived bitterness may be increased or decreased depending on how much effervescent combination is added. Perceived bitterness may also be increased or decreased depending on the ratio of sodium bicarbonate to citric acid used. As mentioned above, the effervescent combination may be added in a ratio of about 2 to about 3 moles sodium bicarbonate to about 1 mole citric acid. In a particular embodiment, increasing the amount of the effervescent combination may decrease the perception of bitterness. For example, an increase in effervescent combination concentration of at least 1%, 2%, 3%, 4%, 5%, 6%, 7%, 8%, 9%, 10%, 20%, 30%, 40%, 50%, 100%, 200%, etc. may result in a decrease in the perception of bitterness. Additionally, or alternatively, adding the effervescent combination in a ratio of about 2:1 to about 3:1 moles of sodium bicarbonate to citric acid may decrease the perception of bitterness. In some embodiments, adding the effervescent combination may increase electric potential by about 1%, 5%, 10%, 25%, 30%, 35%, 40%, 45%, 50%, etc.

Thus, a decreased perception of bitterness may be represented by an increase in electric potential. Alternatively, an increased perception of bitterness may be represented by a decrease in electric potential.

Any suitable method for measuring electric potential of a solution may be used. For example, a carbon dioxide electrode measuring the degree of carbonation/electric potential may be dipped in a solution comprising the molecule or composition to assess degree of carbonation (in mV).

Any carbon dioxide electrode may be used to measure electric potential. Many are commercially available. For example, the ORION™ 9502BNWP carbon dioxide electrode (available from THERMO SCIENTIFIC™) may be used. The 9502BNWP carbon dioxide electrode may be used on any ISE meter with a BNC connection. Additional carbon dioxide electrodes include the ORION™ VERSA STAR PRO™ 4-Star pH/ISE meter or ORION™ 5-Star pH/ISE/DO/conductivity meter (also available from THERMO SCIENTIFIC™).

As shown in the Examples, electric potential readings of compositions/solutions containing the effervescent combination were compared to solutions without the effervescent combination. The inventors determined that when electric potential of the molecule or composition dissolved in water increases, bitterness perception of the molecule or composition decreases.

### F. Additional Embodiments

The section contains non-limiting examples of additional embodiments.

Embodiment 1: An effervescent composition is provided herein comprising:
an effervescent combination comprising a molar ratio of about 2:1 to about 3:1 sodium bicarbonate to citric acid;
an active pharmaceutical ingredient;
and one or more pharmaceutically acceptable excipients.

Embodiment 2: The effervescent composition of embodiment 1, wherein the composition has a positive electric potential of at least about 5 mV when dissolved in 100 mL water and measured at about 25° C.

Embodiment 3: The effervescent composition of embodiment 1 or 2, wherein the effervescent combination is present in the effervescent composition in an amount to increase the electric potential of the composition by at least about 15 mV when the composition is dissolved in 100 mL water and measured at 25° C. compared to the composition dissolved in 100 ml of water at 25° C. without the effervescent combination.

Embodiment 4: The effervescent composition of any one of the previous embodiments, wherein the effervescent combination is present in the composition in an amount from about 20% to about 40% by weight of the composition.

Embodiment 5: The effervescent composition of any one of the previous embodiments, wherein the active pharmaceutical ingredient is an inorganic salt.

Embodiment 6: The effervescent composition of embodiment 5, wherein the inorganic salt is calcium carbonate, zinc sulphate, magnesium chloride, or a combination thereof.

Embodiment 7: The effervescent composition of any one of embodiments 1-4, wherein the active pharmaceutical ingredient is an organic salt.

Embodiment 8: The effervescent composition of embodiment 7, wherein the organic salt is dextromethorphan hydrobromide, naproxen sodium, or a combination thereof.

Embodiment 9: The effervescent composition of any one of the previous embodiments, wherein the one or more pharmaceutically acceptable excipient is a sweetener, a flavoring agent, or a lubricant.

Embodiment 10: The effervescent composition of any one of the previous embodiments, wherein the effervescent composition is an oral dosage form.

Embodiment 11: The effervescent composition of any one of the previous embodiments, wherein the oral dosage form is a solid oral dosage form comprising a tablet, powder, granule, or an extruded form, such as a rod.

Embodiment 12: The effervescent composition of embodiment 10, wherein the effervescent composition is a liquid dosage form, such as a solution.

Embodiment 13: A method of reducing bitterness perception of a molecule or composition, the method comprising:

adding an effervescent combination comprising a molar ratio of about 2:1 to about 3:1 sodium bicarbonate to citric acid to the molecule or composition.

Embodiment 14: The method of embodiment 13, comprising adding the effervescent combination to the molecule or composition in an amount to increase the electric potential of the molecule or composition by at least about 15 mV when dissolved in 100 mL water and measured at 25° C. compared to the molecule or composition when dissolved in 100 mL water and measured at 25° C. without the effervescent combination.

Embodiment 15: The method of embodiments 13 or 14, wherein the molecule or composition comprises an active pharmaceutical ingredient.

Embodiment 16: The method of embodiment 15, wherein the active pharmaceutical ingredient comprises an inorganic salt.

Embodiment 17: The method of embodiment 16, wherein the inorganic salt is calcium carbonate, zinc sulphate, magnesium chloride, or a combination thereof.

Embodiment 18: The method of embodiment 15, wherein the active pharmaceutical ingredient is an organic salt.

Embodiment 19: The method of embodiment 18, wherein the organic salt is dextromethorphan hydrobromide or naproxen sodium.

Embodiment 20: A method of determining bitterness perception of a molecule or composition, the method comprising:
measuring electric potential of the molecule or composition, wherein the molecule or composition is dissolved in water, and
wherein as electric potential increases bitterness perception of the molecule or composition decreases.

Embodiment 21: The method of embodiment 20, further comprising adjusting the bitterness perception of the molecule or composition by adding an effervescent combination comprising citric acid and sodium bicarbonate to the molecule or composition.

Embodiment 22: The method of embodiment 21, wherein the effervescent combination comprises a molar ratio of about 2:1 to about 3:1 sodium bicarbonate to citric acid.

Embodiment 23: The method of any one of embodiments 20-22, wherein the molecule or composition comprises an active pharmaceutical ingredient.

Embodiment 24: An effervescent composition comprising:
an effervescent combination comprising a molar ratio of about 2:1 to about 3:1 sodium bicarbonate to citric acid;
an active pharmaceutical ingredient selected from the group consisting of calcium carbonate, zinc sulphate, magnesium chloride, dextromethorphan hydrobromide, naproxen sodium, acetaminophen, caffeine, and a combination thereof; and
one or more pharmaceutically acceptable excipients;
wherein the effervescent composition is a solid oral dosage form comprising a tablet.

Embodiment 25: The effervescent composition of embodiment 24, wherein the composition has a positive electric potential of at least about 5 mV when dissolved in 100 mL water and measured at about 25° C.

Embodiment 26: The effervescent composition of embodiment 24 or 25, wherein the effervescent combination is present in the effervescent composition in an amount to increase the electric potential of the composition by at least about 15 mV when the composition is dissolved in 100 mL water and measured at 25° C. compared to the composition dissolved in 100 ml of water at 25° C. without the effervescent combination.

Embodiment 27: The effervescent composition of any one of embodiments 25-26, wherein the effervescent combination is present in the composition in an amount from about 20% to about 40% by weight of the composition.

Embodiment 28: The effervescent composition of any one of embodiments 25-27, wherein the one or more pharmaceutically acceptable excipient is a sweetener, a flavoring agent, or a lubricant.

Embodiment 29: A method of determining bitterness perception of a molecule or composition, the method comprising:
measuring electric potential of the molecule or composition, wherein the molecule or composition is dissolved in water;
wherein as electric potential increases bitterness perception of the molecule or composition decreases.

Embodiment 30: The method of embodiment 29, further comprising adjusting the bitterness perception of the molecule or composition by adding an effervescent combination comprising citric acid and sodium bicarbonate to the molecule or composition.

Embodiment 31: The method of embodiment 30, wherein the effervescent combination comprises a molar ratio of about 2:1 to about 3:1 sodium bicarbonate to citric acid.

Embodiment 32: The method of embodiment 30 or 31, comprising adding the effervescent combination to the molecule or composition in an amount to increase the electric potential of the molecule or composition by at least about 15 mV when dissolved in 100 mL water and measured at 25° C. compared to the molecule or composition when dissolved in 100 mL water and measured at 25° C. without the effervescent combination.

Embodiment 33: The method of any one of embodiments 29-32, wherein the molecule or composition comprises an active pharmaceutical ingredient.

Embodiment 34: The method of embodiment 33, wherein the active pharmaceutical ingredient comprises an inorganic salt.

Embodiment 35: The method of embodiment 34, wherein the inorganic salt is calcium carbonate, zinc sulphate, magnesium chloride, or a combination thereof.

Embodiment 36: The method of embodiment 33, wherein the active pharmaceutical ingredient is an organic salt.

Embodiment 37: The method of embodiment 36, wherein the organic salt is dextromethorphan hydrobromide or naproxen sodium.

### EXAMPLES

The following examples are merely illustrative, and do not limit this disclosure in any way.

### Example 1: Formulations

Formulations comprising the effervescent combination (sodium bicarbonate and citric acid) and molecules/compositions that impart a bitter taste (such as APIs or compositions comprising APIs) can be prepared according to Tables 1-5 below. Exemplary formulations according to the disclosure were made according to the following general procedure. 100 mL of deionized water from MILLI-Q® Q-POD® water purifier (having about 1.9 ppb total organic content) was weighed out in an appropriately sized glass beaker. The ingredients mentioned in Tables 1-5 below were weighed out and added to the glass beaker containing deionized water, and then the mixture was stirred for 15 minutes until the blend is homogenous. Each ingredient was sieved through a mesh 18 strainer (mesh 19 strainer size means particles won't be greater than 0.0394 inches (1000 microns or 1 mm) in diameter) to remove clumps before weighing. The METTLER TOLEDO® XP6035S was used to measure ingredient weight, but any suitable weighing balance may be used.

Various combinations of APIs (e.g., calcium carbonate, zinc sulfate, magnesium chloride, etc.) along with the effervescent combination (sodium bicarbonate and citric acid) were prepared in deionized water (100 mL). Formulations not comprising the effervescent combination were also prepared to be included in the electric potential experiment shown in Example 2.

The following tables illustrate the preparation of effervescent formulations including tablets but preparation methods are not limited to these.

TABLE 1
Formulation 1 [Mixed Berry Flavor]

| | MOR | Lerma | | | |
|---|---|---|---|---|---|
| | Material | Material | | | Ingredient |
| Ingredient | # | # | mg/tablet | % w/w | Function |
| Calcium | RM01590 | 88123336 | 1028.000 | 70.459 | API |
| Carbonate | | | | | |
| (VITASMOOTH ®) | | | | | |
| Calcinated Sodium | RM01498 | 86132087 | 200.00 | 13.708 | Alkalinizing |
| Bicarbonate | | | | | agent |
| Citric Acid, | RM01494 | 87054055 | 200.000 | 13.708 | Alkalinizing |
| anhydrous | | | | | agent |
| Stevia | RM01556 | 89519632 | 11.700 | 0.802 | Sweetener |
| EVOSPRAY ® | RM01684 | 89563100 | 4.700 | 0.322 | Flavoring |
| Natural Mixed | | | | | Agents |
| Berry Flavor | | | | | |
| WONF | | | | | |
| Magnesium | RM01484 | 57878308 | 14.600 | 1.001 | Lubricant |
| Stearate | | | | | |
| Total | | | 1459.000 | 100.000 | |

The effervescent combination of sodium bicarbonate and citric acid was combined with mineral calcium carbonate (VITASMOOTH®) to produce a suitable preparation of Formulation 1 as shown in Table 1.

TABLE 2
Formulation 2 [Orange Flavor Formula]

| | MOR | | | | |
|---|---|---|---|---|---|
| | Material | Lerma | | | Ingredient |
| Ingredient | # | Material# | mg/tablet | % w/w | Function |
| Calcium | RM01590 | 88123336 | 1028.000 | 70.459 | API |
| Carbonate | | | | | |
| (VITASMOOTH ®) | | | | | |
| Calcinated Sodium | RM01498 | 86132087 | 200.00 | 13.708 | Alkalinizing |
| Bicarbonate | | | | | agent |
| Citric Acid, | RM01494 | 87054055 | 200.00 | 13.708 | Alkalinizing |
| anhydrous | | | | | agent |
| Stevia | RM01556 | 89519632 | 11.700 | 0.802 | Sweetener |
| EVOSPRAY ® | RM01683 | 89563119 | 4.700 | 0.322 | Flavoring |
| Natural Orange | | | | | Agents |
| Flavor WONF | | | | | |
| Magnesium | RM01484 | 57878308 | 14.600 | 1.001 | Lubricant |
| Stearate | | | | | |
| Total | | | 1459.000 | 100.000 | |

The effervescent combination of sodium bicarbonate and citric acid was combined with mineral calcium carbonate (VITASMOOTH®) to produce a suitable preparation of Formulation 2 as shown in Table 2.

TABLE 3
Formulation 3 [Without Effervescent Couple]
Calcium Carbonate for degree of carbonation analysis with NO Effervescent Couple

| Batch# | 231013A-TK | | | Theoretical |
|---|---|---|---|---|
| Batch size | 100 grams | | Concentration | quantity per |
| Ingredient | Material# | Lot# | (%) | tablet (mg) |
| VITASMOOTH ® | RM01590 | 121910045 | 70.459 | 1028.00 |
| 41 (containing | | | | |
| calcium carbonate) | | | | |
| Calcinated sodium | RM01498 | X25AEZ | 0.00 | 0.00 |
| bicarbonate | | | | |
| Citric acid, | RM01494 | X24BU5 | 0.00 | 0.00 |
| anydrous | | | | |
| Stevia | RM01556 | ZE22E00601 | 0.802 | 11.70 |
| EVOSPRAY ® | RM01683 | SD22-2306-1 | 0.322 | 4.70 |
| Natural Orange | | | | |
| Flavor WONF | | | | |
| Magnesium | RM01484 | X248BB | 1.001 | 14.60 |
| stearate | | | | |

To produce a suitable preparation of Formulation 3 as shown in Table 3, the effervescent combination of sodium bicarbonate and citric acid was not included. The mineral calcium carbonate (VITASMOOTH® 41) was included.

TABLE 4
Formulation 4 [Minerals (Zinc Sulfate &
Magnesium Chloride) without Effervescent Couple]
Minerals (Calcium Carbonate with Zinc Sulfate &
Magnesium Chloride) with NO Effervescent Couple

| Batch# | 230316A-TK | | | Theoretical |
|---|---|---|---|---|
| Batch size | 100 grams | | Concentration | quantity per |
| Ingredient | Material# | Lot# | (%) | tablet (mg) |
| VITASMOOTH ® | RM01590 | 121910045 | 70.459 | 1028 |
| 41 (containing | | | | |
| calcium carbonate) | | | | |
| Calcinated sodium | RM01498 | X25AEZ | 0.00 | 0.00 |
| bicarbonate | | | | |
| Citric acid, | RM01494 | X24BU5 | 0.00 | 0.00 |
| anhydrous | | | | |
| Stevia | RM01556 | ZE22E00601 | 0.802 | 11.7 |
| EVOSPRAY ® | RM01683 | SD22-2306-1 | 0.322 | 4.7 |
| Natural Orange | | | | |
| Flavor WONF | | | | |
| Magnesium | RM01484 | X248BB | 1.001 | 14.6 |
| stearate | | | | |
| Zinc sulfate | | Acros organics | 0.5 | 7.3 |
| | | lot# A0425630 | | |
| Magnesium | | Fisher Scientific | 4.7 | 68.57 |
| chloride | | M35-500, lot# | | |
| heptahydrate | | 181641 | | |

To produce a suitable preparation of Formulation 4 as shown in Table 4, the effervescent combination of sodium bicarbonate and citric acid was not included. The minerals calcium carbonate (VITASMOOTH® 41), zinc sulfate, and magnesium chloride were included.

TABLE 5
Formulation 5 [Minerals (Zinc Sulfate &
Magnesium Chloride) with Effervescent Couple]
Minerals (Calcium Carbonate with Zinc Sulfate & Magnesium Chloride)

| Batch# | 230316B-TK | | | Theoretical |
|---|---|---|---|---|
| Batch size | 100 grams | | Concentration | quantity per |
| Ingredient | Material# | Lot# | (%) | tablet (mg) |
| VITASMOOTH ® | RM01590 | 121910045 | 70.459 | 1028.00 |
| 41 (containing | | | | |
| calcium carbonate) | | | | |
| Calcinated sodium | RM01498 | X25AEZ | 13.708 | 200.00 |
| bicarbonate | | | | |
| Citric acid, | RM01494 | X24BU5 | 13.708 | 200.00 |
| anhydrous | | | | |
| Stevia | RM01556 | ZE22E00601 | 0.802 | 11.70 |
| EVOSPRAY ® | RM01683 | SD22-2306-1 | 0.322 | 4.70 |
| Natural Orange | | | | |
| Flavor WONF | | | | |
| Magnesium | RM01484 | X248BB | 1.001 | 14.60 |
| stearate | | | | |
| Zinc sulfate | | Acros organics | 0.500 | 7.30 |
| | | lot# A0425630 | | |
| Magnesium | | Fisher Scientific | 4.700 | 68.57 |
| chloride | | M35-500, lot# | | |
| heptahydrate | | 181641 | | |

To produce a suitable preparation of Formulation 1 as shown in Table 1, the effervescent combination of sodium bicarbonate and citric acid along was included along with minerals calcium carbonate (VITASMOOTH® 41), zinc sulfate, and magnesium chloride.

### Example 2: Electric Potential Experiment

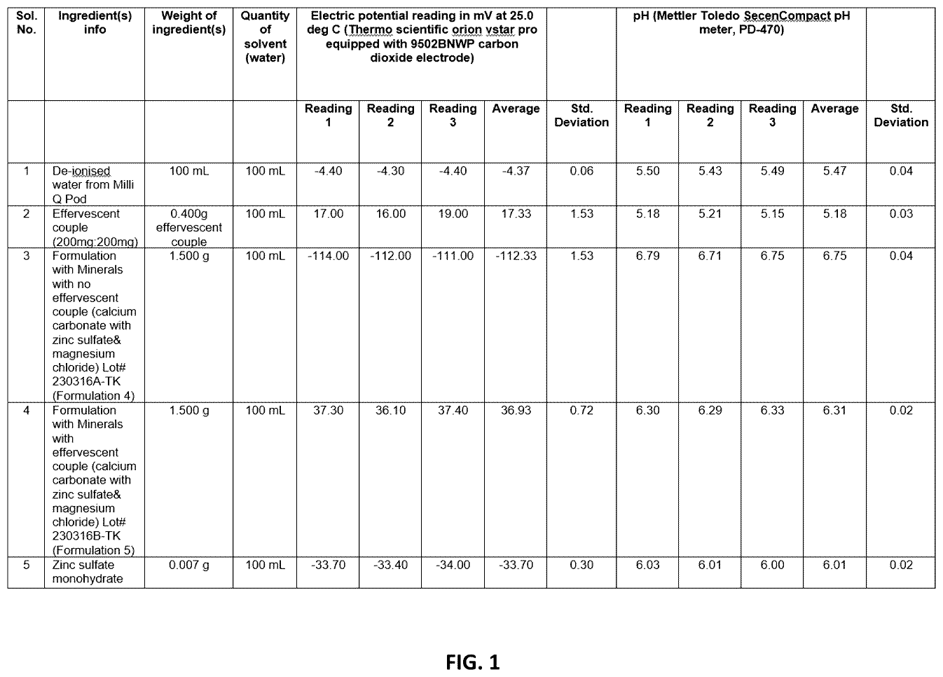

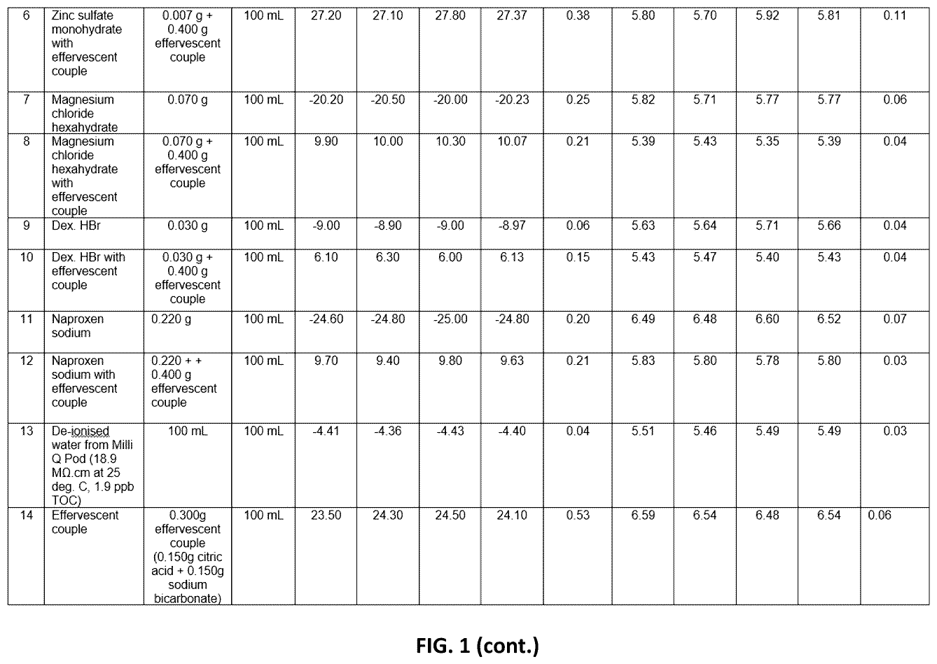

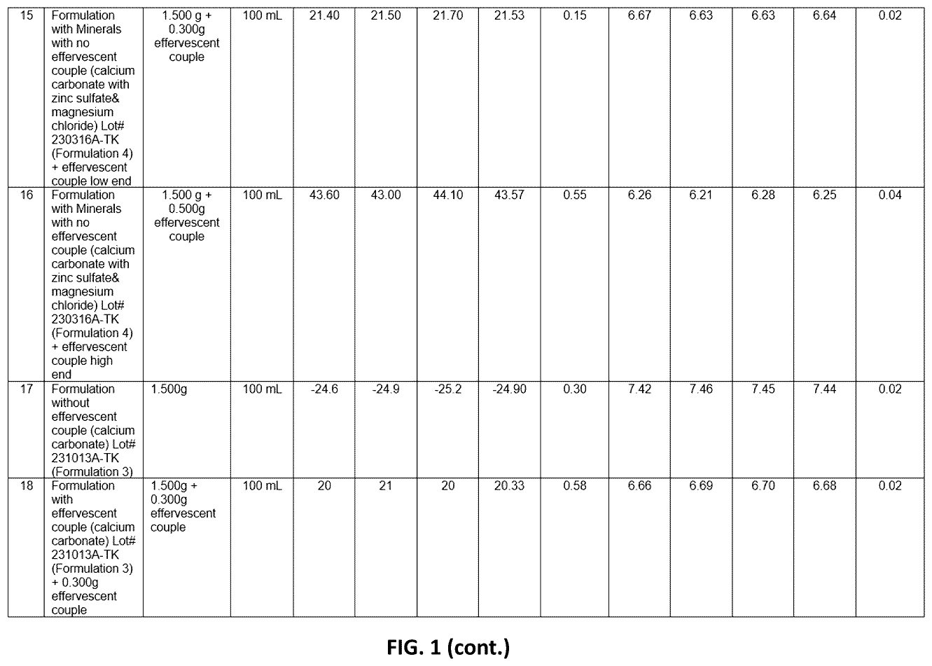

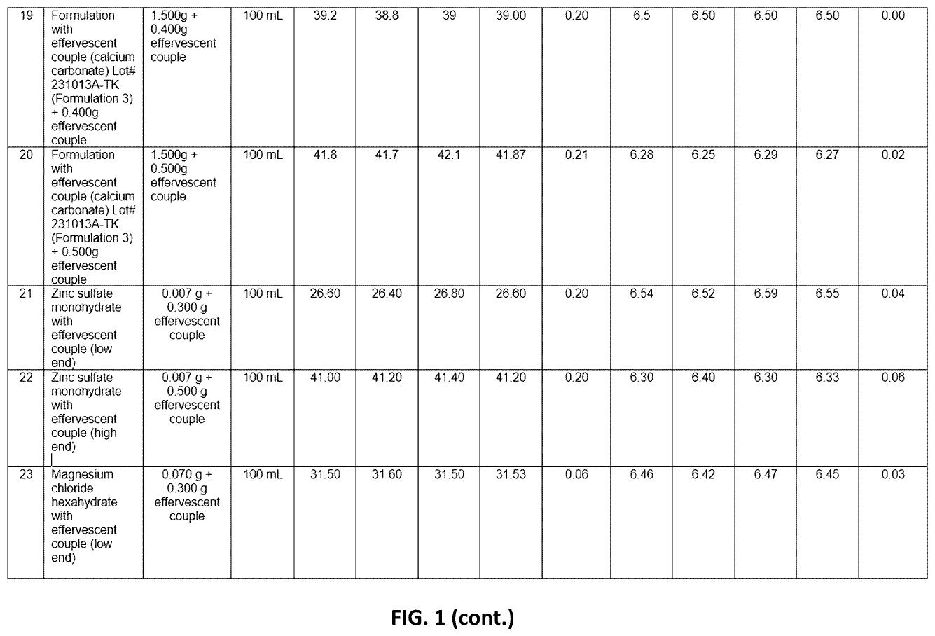

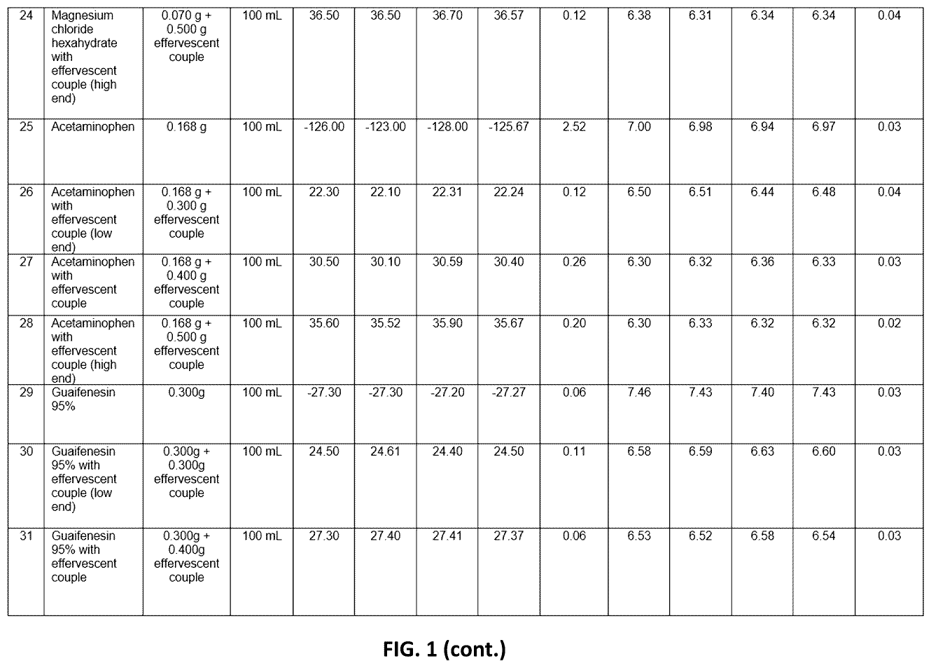

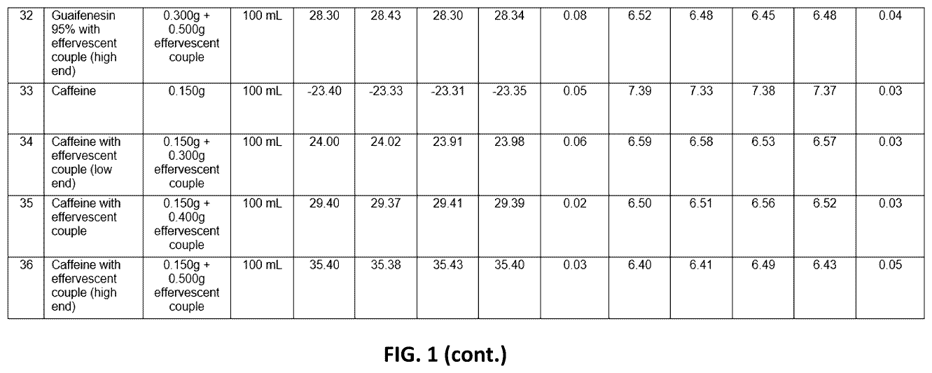

Various combinations of bitter tasting molecules/compositions (e.g., APIs or compositions comprising APIs) along with the effervescent combination were prepared as solutions (see Sol. Nos. 1-36 in FIG. 1) per the general procedure described in Example 1. For the electric potential experiment, after the solutions were stirred, a carbon dioxide electrode (which measures the degree of carbonation) was dipped in the glass beaker to assess degree of carbonation (unit is electric potential in mV). The electric potential (in mV) and pH of example solutions were measured, and the readings are listed in FIG. 1. The THERMO SCIENTIFIC™ ORION™ VSTAR PRO™ 9502BNWP carbon dioxide electrode was used to measure electrical potential (mV). The METTLER TOLEDO SEVENCOMPACT™ PH meter PD-470 was used to measure the pH. Readings were noted at about a 5 minute time point when the reading was stabilized. The same procedure was repeated for all solutions shown in FIG. 1. The carbon dioxide electrode was rinsed with deionized water and cleaned with a lint free tissue in between samples. The amount of the effervescent combination was kept constant through various combinations unless the weight was changed as indicated in FIG. 1, and blank readings were also taken for the effervescent combination only and the deionized water only.

As shown in FIG. 1, all solutions including the effervescent combination (sodium bicarbonate and citric acid) had an increase in electric potential (mV) compared to those that did not include the effervescent combination. For example, Sol. No. 1 only includes deionized water, which resulted in an average electric potential of −4.37 mV. Sol. No. 2 includes the effervescent combination in a ratio of 200 mg: 200 mg, which resulted in an average electric potential of 17.33 mV. The same can be seen for all solutions containing various APIs (i.e., solutions containing calcium carbonate, zinc sulfate, magnesium chloride, Dex. HBr, naproxen sodium, acetaminophen, guaifenesin, caffeine, and combinations thereof) with and without the effervescent combination. For example, Sol. No. 3 (which corresponds to Formulation 4 in Example 1), which includes a solution containing calcium carbonate, zinc sulfate, and magnesium chloride, but does not contain the effervescent combination, has an average electric potential of −122.33 mV. However, Sol. No. 4 (which corresponds to Formulation 5 in Example 1), which includes the same APIs and also includes the effervescent combination, has an average electric potential of 36.93 mV.

FIG. 1 also shows that increasing the weight of the effervescent combination increases the electric potential reading. For example, Sol. Nos. 26-28 (solutions containing acetaminophen as the API) show that including 0.300 g of the effervescent combination (low end) results in an average electric potential of 22.23567 mV, including 0.400 g of the combination results in an average of 30.39567 mV, and including 0.500 g (high end) of the combination results in an average of 35.67333 mV.

As can be seen from the electric potential (mV) readings in FIG. 1, there is a significant change in the electric potential from negative electric potential to positive electric potential, demonstrating a change in the bitterness profile of the model formulation. A negative electric potential (−mV) denotes a more bitter taste perception, whereas a positive electric potential (+mV) signifies a less bitter taste perception. Thus, the effervescent combination of sodium bicarbonate and citric acid helps mask the unpleasant bitter taste perception.

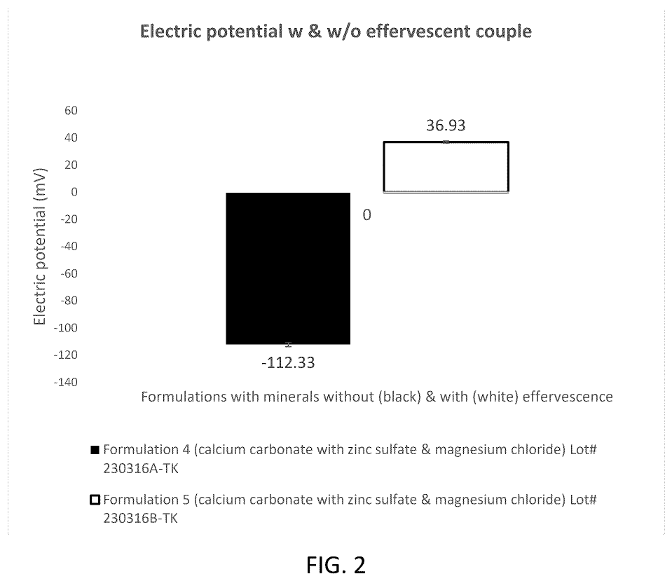

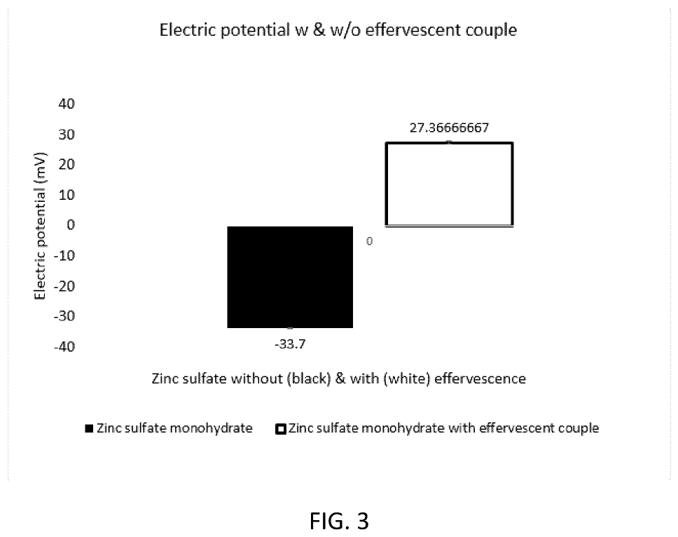

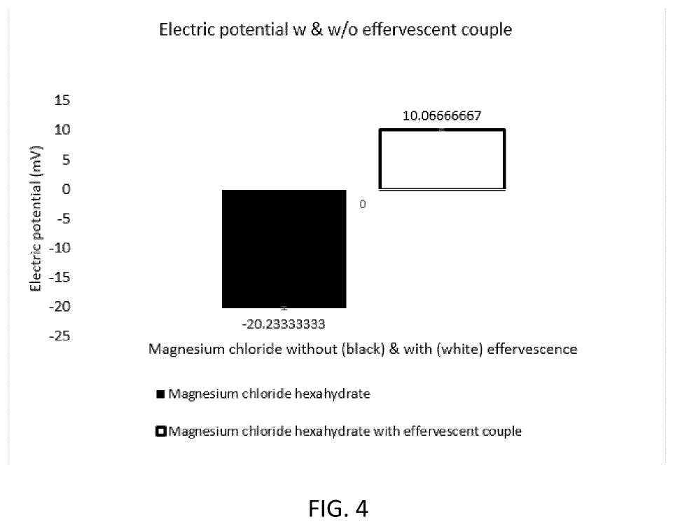

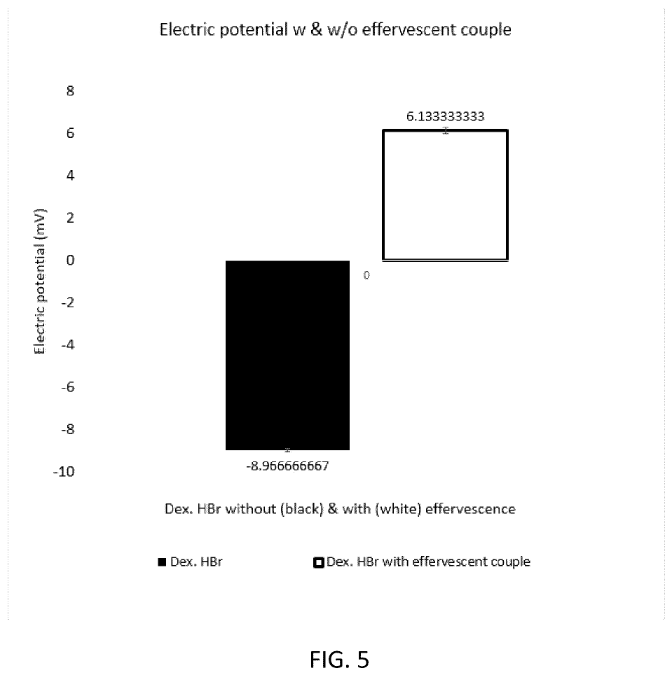

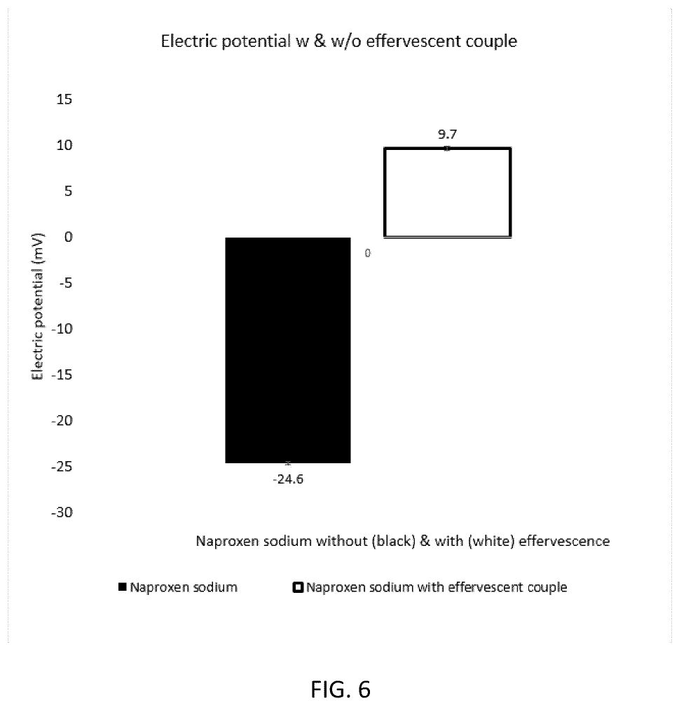

FIGS. 2-6 (bar charts) show the electric potential (mV) for solutions containing various combinations of APIs (i.e., combinations of calcium carbonate, magnesium chloride, zinc sulfate, Dex. HBr, and naproxen sodium) with or without the effervescent combination. Like FIG. 1, each of FIGS. 2-6 indicate that the solutions containing the effervescent combination show higher, positive electric potential readings. For example, FIG. 2 shows that a solution containing minerals (calcium carbonate, zinc sulfate, and magnesium chloride) with no effervescent combination (which corresponds to Formulation 4) has an electric potential of −122.33 mV, while a solution containing minerals (calcium carbonate, zinc sulfate, and magnesium chloride) with the effervescent combination (which corresponds to Formulation 5) has an electric potential of 36.93 mV.

Additionally, the pH drop in all solutions containing the effervescent combination signifies that there is a formation of carbonic acid which delays the initial cell depolarization referred to as receptor potential, thereby masking the bitter taste perception of ingredients such as calcium carbonate, zinc sulfate, magnesium chloride, Dex. HBr, naproxen sodium, etc.

What is claimed is:

1. An effervescent composition comprising:
an effervescent combination comprising a molar ratio of about 2:1 to about 3:1 sodium bicarbonate to citric acid;
an active pharmaceutical ingredient selected from the group consisting of calcium carbonate, zinc sulphate, magnesium chloride, dextromethorphan hydrobromide, naproxen sodium, acetaminophen, caffeine, and a combination thereof; and
one or more pharmaceutically acceptable excipients;
wherein the effervescent composition is a solid oral dosage form comprising a tablet.

2. The effervescent composition of claim 1, wherein the composition has a positive electric potential of at least about 5 mV when dissolved in 100 mL water and measured at about 25° C.

3. The effervescent composition of claim 1, wherein the effervescent combination is present in the effervescent composition in an amount to increase the electric potential of the composition by at least about 15 mV when the composition is dissolved in 100 mL water and measured at 25° C. compared to the composition dissolved in 100 mL of water at 25° C. without the effervescent combination.

4. The effervescent composition of claim 1, wherein the effervescent combination is present in the composition in an amount from about 20% to about 40% by weight of the composition.

5. The effervescent composition of claim 1, wherein the one or more pharmaceutically acceptable excipient is a sweetener, a flavoring agent, or a lubricant.

6. A method of determining bitterness perception of a molecule or composition, the method comprising:
measuring electric potential of the molecule or composition, wherein the molecule or composition is dissolved in water;
wherein as electric potential increases bitterness perception of the molecule or composition decreases.

7. The method of claim 6, further comprising adjusting the bitterness perception of the molecule or composition by adding an effervescent combination comprising citric acid and sodium bicarbonate to the molecule or composition.

8. The method of claim 7, wherein the effervescent combination comprises a molar ratio of about 2:1 to about 3:1 sodium bicarbonate to citric acid.

9. The method of claim 7, comprising adding the effervescent combination to the molecule or composition in an amount to increase the electric potential of the molecule or composition by at least about 15 mV when dissolved in 100 mL water and measured at 25° C. compared to the molecule or composition when dissolved in 100 mL water and measured at 25° C. without the effervescent combination.

10. The method of claim 6, wherein the molecule or composition comprises an active pharmaceutical ingredient.

11. The method of claim 10, wherein the active pharmaceutical ingredient comprises an inorganic salt.

12. The method of claim 11, wherein the inorganic salt is calcium carbonate, zinc sulphate, magnesium chloride, or a combination thereof.

13. The method of claim 10, wherein the active pharmaceutical ingredient is an organic salt.

14. The method of claim 13, wherein the organic salt is dextromethorphan hydrobromide or naproxen sodium.
